(19) United States
(12) Patent Application Publication
(10) Pub. No.: US 2006/0269781 A1
(43) Pub. Date: Nov. 30, 2006

(54) Diarylamino substituted compounds and an electroluminescent device having the compounds

(76) Inventors: Jun-Liang Lai, Chiayi Hsien (TW); Kuo-Wei Huang, Taipei (TW)

Correspondence Address: POSZ LAW GROUP, PLC, 12040 SOUTH LAKES DR., SUITE 101, RESTON, VA, 20191, US

(21) Appl. No.: 11/135,400

(22) Filed: May 24, 2005

(51) Int. Cl. H01L 51/54; C07C 211/00; H05B 33/12

(52) U.S. Cl. 428/690; 428/917; 313/504; 313/506; 257/E51.051; 564/426; 564/427; 564/428; 564/429; 564/434

(57) ABSTRACT

Diarylamino substituted compounds have the following representative formula (1): 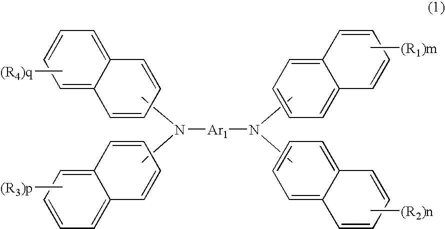
wherein Ar₁ is an aromatic group containing 6-30 carbon atoms; R₁, R₂ R₃ and R₄ are identical or different substituents selected from the group consisting of alkyl containing 1-4 carbon atoms, alkoxyl containing 1-4 carbon atoms, sulfoalkyl containing 1-4 carbon atoms, dialkylamine containing 1-4 carbon atoms and halogen atom; m, n, p and q are integers ranging from 0-2. The diarylamino substituted compounds can be sublimated at a temperature more than 200° C. in vacuum environment. Without forming a liquid phase, the diarylamino substituted compounds can be cleaned out conveniently. Moreover, by using the diarylamino substituted compounds as organic material in the layers, the organic electroluminescent device has high luminescent efficiency and excellent thermal resistance so that the life-time and quality of the organic electroluminescent device are greatly improved.

### BACKGROUND OF THE INVENTION

1. Field of the Invention

The present invention relates to organic light emitting compounds, and more particularly to diarylamino substituted compounds that have excellent thermal stability.

2. Description of Related Art

An organic light-emitting diode (OLED) is composed of a cathode, an anode and multiple electroluminescent layers. The electroluminescent layers contain special organic materials respectively being a hole-injecting material, a hole-transmitting material, an electroluminescent material, an electron-conducting material and electron-injecting material. All electroluminescent layers are formed between the cathode and the anode by thermal vapor deposition or spin coating. When voltage is applied to the organic light-emitting diode, the anode generates holes and the cathode generates electrons. When the holes and the electrons combine with each other in the electroluminescent layers, light with a color is created and is emitted out of the organic light-emitting diode. The color of the light can be changed by using different organic light-emitting materials.

Organic light-emitting diodes have many excellent properties such as self-luminance, low thickness, wide view, and quick response so that the organic light-emitting diode is regarded as the most significant prospect in the next generation of electronic display development. However, the organic light-emitting diode has a critical defect of having short life-time in use such that it has many limitations in application. This defect is particularly serious in an otherwise very promising device and needs to be resolved as soon as possible.

Factors limiting application of the organic light-emitting diode are various, but some relating to organic materials are:
1. Poor packaging: the organic materials interact with oxygen and moisture to deteriorate.
2. Poor stability of the organic materials: the luminescent layers made of the organic materials easily generate crystallization or deteriorate.
3. Photo-sensitive: the organic materials will deteriorate once they are exposed to sunlight.

For example, 4-cyanocoumarin is one kind of organic material that is photo-sensitive and its purity and luminescent efficiency are influenced by sunlight so that 4-cyanocoumarin is difficult to apply to an organic light-emitting diode in reality.

Another kind of organic material is N,N′-Di(naphthalene-1-yl)-N,N′-diphenyl-benzidine (NPB) that is applied in hole-transporting layer and has Tg at 96° C. only. Therefore, the organic light-emitting diode made of the NPB can not endure a temperature more than 96° C. and can not be applied to certain products such as a panel specially applied to vehicle.

In order to find a new material to substitute NPB to resolve this problem, many manufacturers are dedicated to develop new materials. At present, several triarylamine compounds are discovered to improve thermal stability. However, the triarylamine compounds still have some drawbacks. Because the triarylamine compounds are melted and then vaporized to deposit on a glass substrate during vapor deposition, the triarylamine compounds easily deteriorate in liquid phase. If the triarylamine compounds have high molecular weight and need high heating temperature to vaporize, the deteriorating chance is greatly increased. Moreover, cleaning equipment stained by the vaporized triarylamine compounds is difficult.

Kodak™ Company has disclosed a series of aromatic compounds to serve as hole-transporting material. However, those disclosed aromatic compounds have poor hole-transporting efficiency.

Except hole-transporting materials, other materials have the same development that uses a high thermal stability material to substitute a low thermal stability one. One example is to use C-545T (10-(2-benzothiazolyl)-1,1,7,7,-tetramethyl-2,3,6,7-tetrahydro-1H,5H,11H-[1]benzopyrano[6,7,8-ij]quinolizin-11-one] to replace another type of coumarin C-6 (3-(2′-benzothiazolyl)-7-diethylaminocoumarin) to serve as green dopant. Another example is to use diarylvinyl compounds to replace DPVBi (4,4′-bis(2,2′-diphenylvinyl)-1,1′-biphenyl). According to these two examples, it is clear that improving thermal stability of the organic material positively extends the life-time of the organic light-emitting diode.

The present invention has arisen to provide novel materials having excellent properties to substitute the conventional organic materials and to improve quality of the organic light-emitting diode.

### SUMMARY OF THE INVENTION

One main objective of the present invention is to provide a series of diarylamino substituted compounds that have excellent thermal stability to improve the life-time of the organic light-emitting diodes.

To achieve the foregoing objective, the diarylamino substituted compounds have the following representative formula (1):

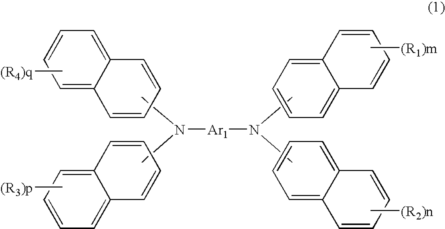

wherein Ar₁ is an aromatic group containing 6-30 carbon atoms; R₁, R₂, R₃ and R₄ are identical or different substituents selected from the group consisting of alkyl containing 1-4 carbon atoms, alkoxyl containing 1-4 carbon atoms, sulfoalkyl containing 1-4 carbon atoms, dialkylamine containing 1-4 carbon atoms and halogen atom; m, n, p and q are integers ranging from 0-2.

Another main objective of the present invention is to provide an organic electroluminescent device that contains the foregoing diarylamino substituted compounds of formula (1), wherein the diarylamino substituted compounds are selectively applied in a hole-injecting layer, a hole-transporting layer or a luminescent layer.

By using the diarylamino substituted compounds as organic material in the layers, the organic electroluminescent device has high luminescent efficiency and excellent thermal resistance so that the life-time and quality of the organic electroluminescent device are greatly improved. Moreover, the diarylamino substituted compounds can be sublimated in a high-pressure vacuum system to eliminate the melting process to liquid phase so that the residual compounds are easily cleaned out from a crucible. Therefore, the cleaning process of used equipment after manufacture of the organic electroluminescent device becomes much more convenient than in prior art. Further benefits and advantages of the present invention will become apparent after a careful reading of the detailed description.

### DETAILED DESCRIPTION OF THE PREFERRED EMBODIMENT

Diarylamino substituted compounds in accordance with the present invention are particularly applied for manufacture of organic light-emitting diodes and have the following representative formula (1):

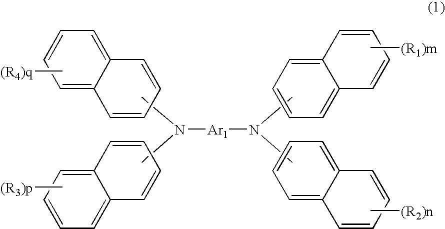

wherein Ar₁ is an aromatic group containing 6-30 carbon atoms; R₁, R₂, R₃ and R₄ are identical or different substituents selected from the group consisting of alkyl containing 1-4 carbon atoms, alkoxyl containing 1-4 carbon atoms, sulfoalkyl containing 1-4 carbon atoms, dialkylamine containing 1-4 carbon atoms and halogen atom; m, n, p and q are integers ranging from 0-2.

The diarylamino substituted compounds have excellent thermal stability and highest occupied molecular orbital (HOMO) energy level so that the diarylamino substituted compounds are suitable for organic material in hole-injecting layer and hole-transporting layer.

The contemporary organic electroluminescent devices that enable diarylamino substituted compounds to be obtained are commonly and sequentially consisted of a substrate, an anode, a hole-injecting layer, a hole-transporting layer, a luminescent layer, an electron-transmitting layer, an electron-injecting layer and a cathode or sequentially a substrate, an anode, a hole-transporting layer, a luminescent layer, an electron-transmitting layer and a cathode.

When the diarylamino substituted compounds are applied to the luminescent layer, the organic electroluminescent device generates visible light with 400-700 nm wavelength. When the diarylamino substituted compounds are used as dopant, the diarylamino substituted compounds take 0.1-50 (w/w %) weight proportion base on the total weight of the luminescent layer. The diarylamino substituted compounds have excellent thermal stability, high luminescent efficiency and are suitable for organic electroluminescent devices emitting blue light, green light and red light to ensure the organic electroluminescent devices having high luminescent efficiency, high color purity, and long life-time.

Additionally, when the diarylamino substituted compounds are applied in the hole-injecting layer or the hole-transporting layer, dopant in the emitting layers is preferred to be the following compounds having the representative formula (2):

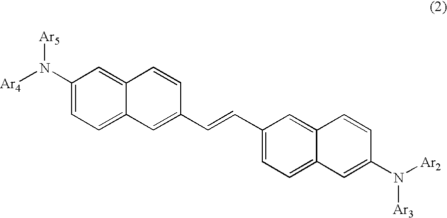

wherein Ar₂, Ar₃, Ar₄, Ar₅ are aromatic groups containing 6-15 carbon atoms.

By adding the compounds of formula (2) into the emitting layer, the organic electroluminescent device emits blue light and has excellent luminescent efficiency and high color purity.

Because the diarylamino substituted compounds in formula (1) have higher molecular weight and more rigid structures in comparison with conventional organic luminescent materials, the melting point and the glass transition temperature (Tg) of the diarylamino substituted compounds are correspondingly higher and thermal resistance of the diarylamino substituted compounds is better than the conventional ones. Moreover, because structures of the diarylamino substituted compounds are non-planar and molecular weights of the diarylamino substituted compounds defined in the formula (1) are proper, the diarylamino substituted compounds can be sublimated in a vacuum heating system to become gas before the system temperature reaches the melting points of the diarylamino substituted compounds.

One preferred embodiment of the diarylamino substituted compound is di-naphthyl substituted compound, wherein one N atom on each di-naphthyl substituted compound combines with α-C and β-C of two naphthyl groups or two N atoms on each di-naphthyl substituted compound respectively combine with α-C and β-C of two naphthyl groups. Moreover, substitutes on the naphthyl group are preferred to be methyl, isobutyl, methoxyl, fluorine or non-substitute. Number of the substituent on the naphthyl group is preferred to be 1 or none.

The Ar₁ group in the formula (1) is an aromatic group containing 6-30 carbon atoms and operationally selected but not limited from the group consisting of:

| | | |
|---|---|---|
| 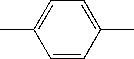 | 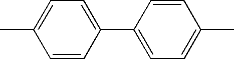 | 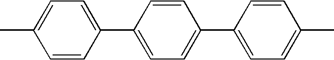 |
| 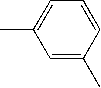 | 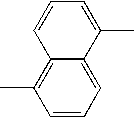 | 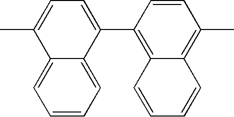 |
| 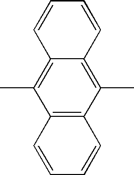 | 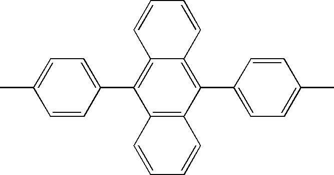 | 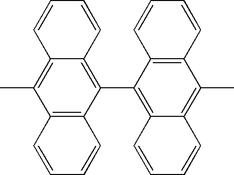 |
| 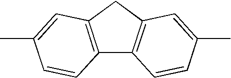 | 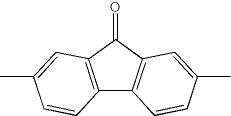 | 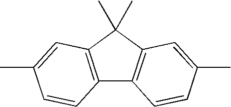 |
| 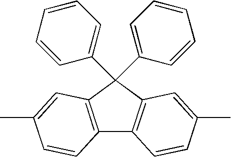 | 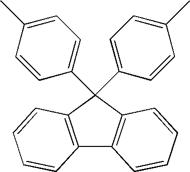 | 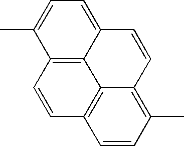 |
| 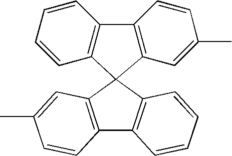 | 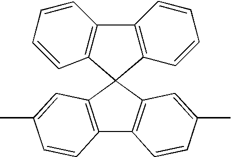 | 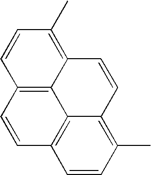 |

Thermal stability of the diarylamino substituted compounds in formula (1) relates to the chemical structure of Ar₁, wherein when the chemical structure of Ar₁ is larger, the thermal stability of the diarylamino substituted compounds is higher. For example, Ar₁ of biphenyl has a better thermal stability than Ar₁ of phenyl. When the chemical structure of Ar₁ is more rigid, the thermal stability of the diarylamino substituted compounds is higher. For example, Ar₁ of fluorene has a better thermal stability than Ar₁ of biphenyl. Additionally, the Ar₁ group further contains at least one substitute such as alkyl, alkoxyl, sulfoalkyl, and dialkylamino group. Another relative factor to the thermal stability is symmetry. When the symmetry degree is higher, the thermal resistance is better.

Preferably, each diarylamino substituted compound has a molecular weight less than 1000, a melting point ranging from 200 to 500° C., and a glass transition temperature (Tg) higher than 80° C. The diarylamino substituted compounds can be heated to sublimate in a vacuum environment to form a transparent membrane that stably exists at room temperature.

The diarylamino substituted compounds are manufactured using present technology by coupling reaction with palladium or copper catalyst and then are further purified by column chromatography, re-crystallization, and sublimation to achieve 99% purity.

Moreover, the diarylamino groups are color-enhancing functional groups to efficiently improve luminescent efficiency in organic materials and to make the emitting light have a wavelength longer than other organic materials having no diarylamino group. Changing the wavelength of emitting light is so-called red-shift phenomenon. By selecting different Ar₁ groups, the diarylamino substituted compounds are enabled to emit lights with different wavelengths in the organic electroluminescent devices. When the Ar₁ is selected from the group consisting of meta-benzol, para-benzol, naphthyl, biphenyl, binaphthyl, fluorene, fluorenone, 9,9-dialkylfluorene, 9,9-diaromatic fluorene, 9,9-spiro bifluorene, pyrene, stilbene, dinaphthyl ethylene and 9,10-diphenyl anthracene, the diarylamino substituted compounds can be dissolved in solvents of toluene, xylene, dichloro methane, and tetrahydrofuran (THF) and emit blue light under ultraviolet lamps. The prefixes meta-(m-) and para-(p-) indicate locations of the nitrogen atoms attached to the Ar₁ group in the form of a benzene ring.

When the Ar₁ is selected from the group consisting of polyaromatic hydrocarbon compounds such as anthracene, bianthracene, tetracene, and pentacene, the diarylamino substituted compounds can be dissolved in solvents of toluene, xylene, dichloro methane, and tetrahydrofuran (THF) and emits green light, yellow light, orange light, or red light under ultraviolet lamps.

Application of the diarylamino substituted compounds in the organic electroluminescent device is illustrated in the following:

One of the diarylamino substituted compounds is heated to 200-500° C., sublimated in a vacuum environment (<10⁻³ torr), and attached to a substrate to compose a membrane. The thickness of the membrane is monitored by a quartz oscillator. The thermal stability of the membrane is determined by thermal resistance and the glass transition temperature (Tg) of the diarylamino substituted compound. When operational temperature is higher than Tg of the diarylamino substituted compound, the membrane deforms and loses its flatness. Because the diarylamino substituted compounds in the present invention have high Tg, thermal resistance of the membrane is significantly improved.

Additionally, the membrane is examined by an analyzer (AC2) to confirm that the diarylamino substituted compounds have highest occupied molecular orbital (HOMO) energy level. However, the HOMO energy levels of the diarylamino substituted compounds change with the variation of Ar₁ groups. Generally speaking, the HOMO energy levels of organic materials in the organic electroluminescent devices should not have great difference therebetween, otherwise, movements of electron charges will be influenced. The HOMO energy levels of the diarylamino substituted compounds of formula (1) range within 5.0-6.0 eV.

Preferred embodiments of the diarylamino substituted compounds applied in the hole-injecting layer and the hole-transporting layer are shown as the following:

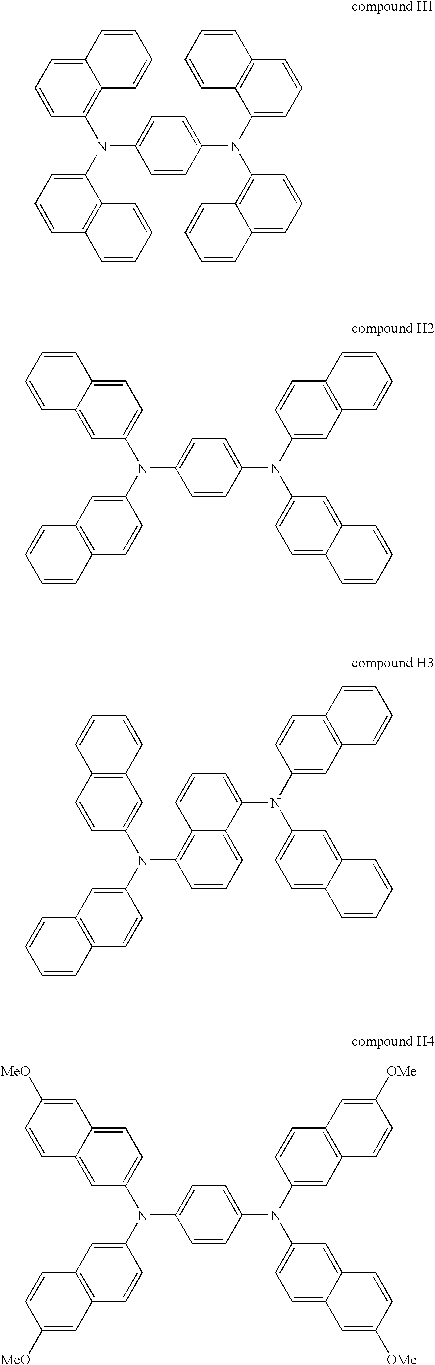

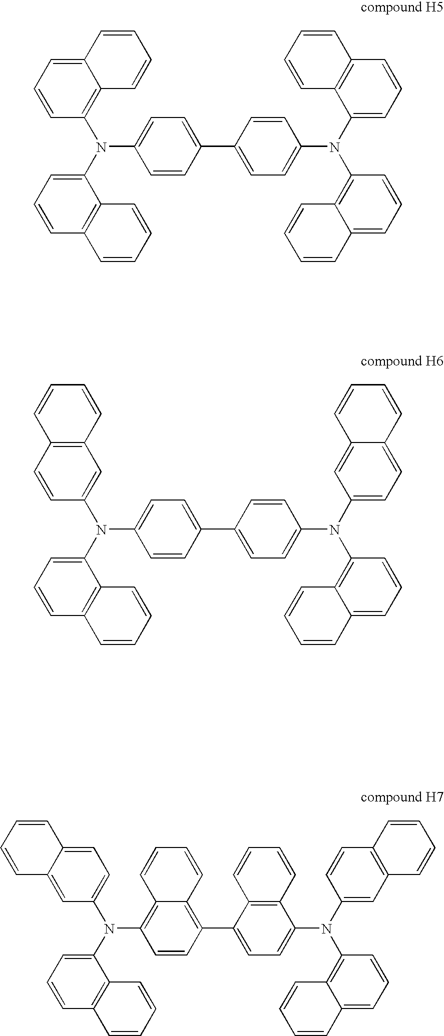

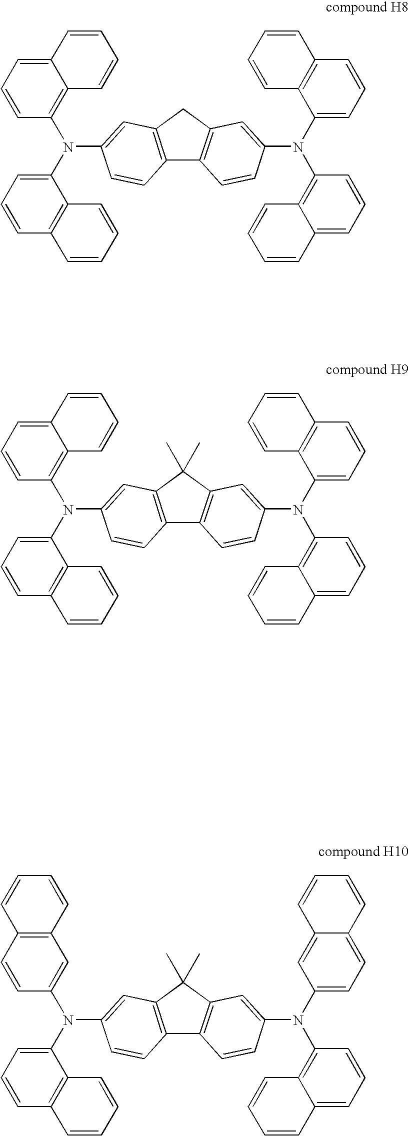

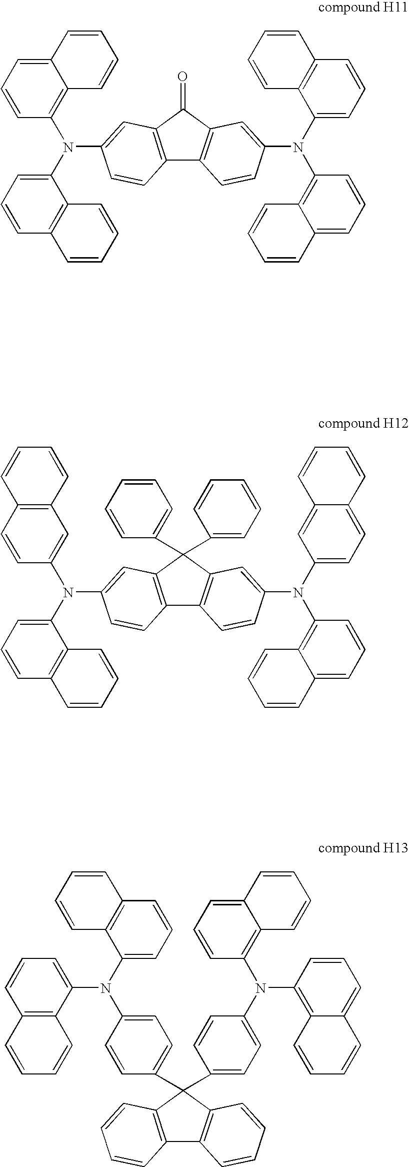

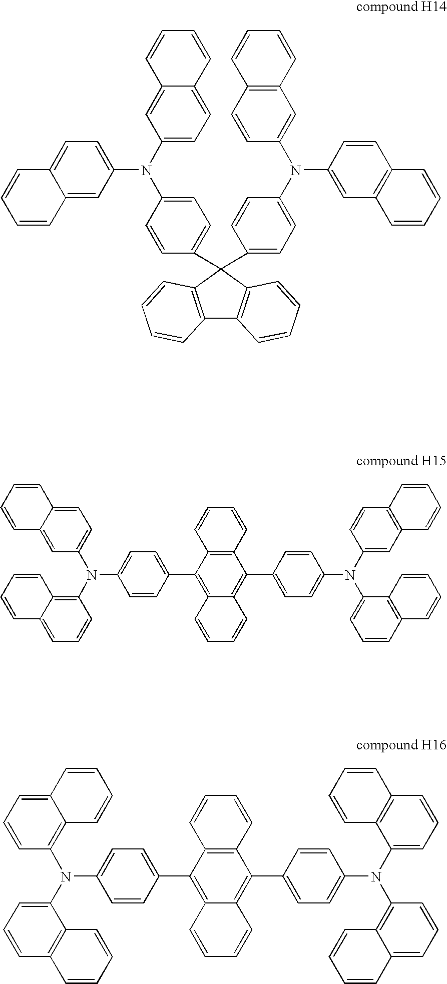

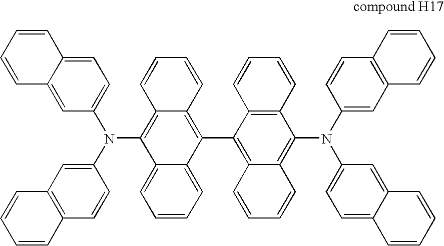

Membrane thickness of the diarylamino substituted compounds in the hole-injecting layer and hole-transporting layer is preferably 5-100 nm. The membrane thickness of the diarylamino substituted compounds influences activating voltages to the organic electroluminescent device. Therefore, the activating voltages can be lowered by adjusting the membrane thickness.

When the diarylamino substituted compounds in formula (1) are applied in the luminescent layer, the diarylamino substituted compounds must be good for hole-carrying efficiency that certainly repulses electron barrier. Therefore, diarylamino substituted compounds are preferred to be taken as a dopant material in the luminescent layer but not a host material. The preferred embodiments of the diarylamino substituted compounds of formula (1) in the luminescent layer are H8, H9, H10, H12, and H17 as shown above.

Additionally, the compounds of formula (2) have the following preferred embodiments:

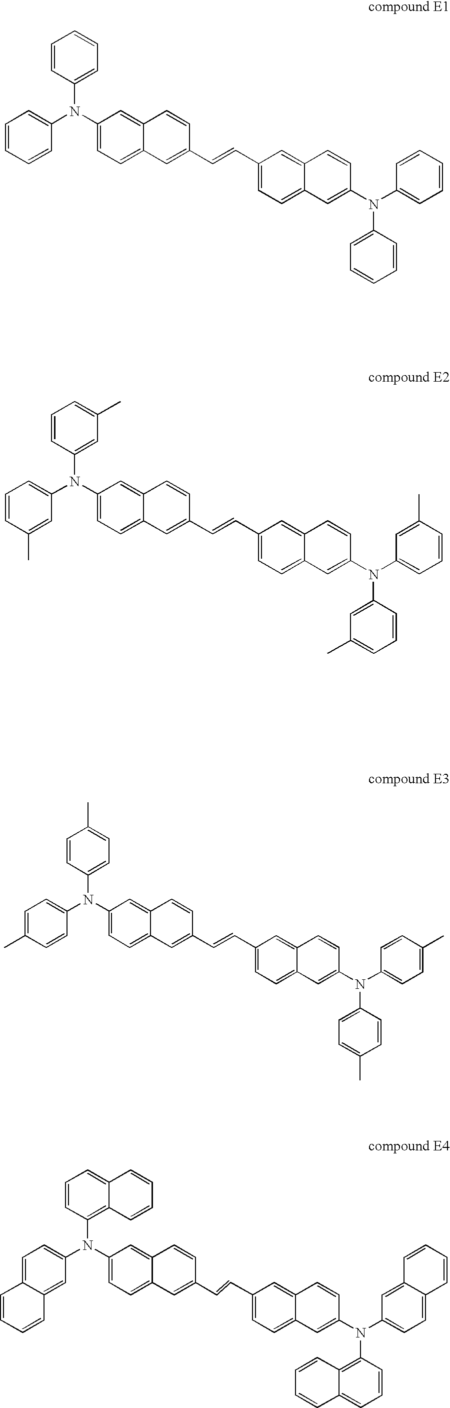

The host material in the luminescent layer is preferably polyaromatic hydrocarbon compounds such as 9,10-bi(2-naphthyl)anthracene, 10,10′-bi(p-biphenyl)-9,9′-bianthracene or metallic complex (for example: Alq₃).

The dopant of formula (1) or formula (2) takes weight proportion of 0.1-50 % based on the host material, preferably 1-10%. The thickness of the luminescent layer is 5-100 nm.

The electron-transmitting layer is made of metal-quinoline complex, l triazine or oxadiazo compounds and has a thickness of 1-100 nm. Because the metal-quinoline complex has excellent thermal stability and can be sublimated under high temperature vacuum environment, it is a preferable material for the electron-transmitting layer. Actual examples are Alq₃, BeBq_{2.}

The electron-injecting layer is made of metal or inorganic ion compounds (such as lithium fluoride orcesium) and has a thickness less than 1 nm.

The cathode is made of metal or metal alloy with low operation voltage such as Al, Li, Mg, Ag, Al—Li alloy, and Mg—Ag alloy and has a thickness of 50-500 nm.

By using the diarylamino substituted compounds in formula (1) in the hole-injecting layer, the hole-transporting layer and the luminescent layer, the organic electroluminescent device is enabled to emit stably and continuously after electrical connection.

### EXAMPLES FOR SYNTHESIZING THE DIARYLAMINO SUBSTITUTED COMPOUNDS

### Example 1

### Synthesization of H1

First, 10 g of p-phenylenediamine, 112.77 g of 1-iodonaphthalene, 25.94 g of potassium hydroxide, 0.37 g of cupreous chloride, 0.67 g of 1,10-phenanthroline and 185 ml of toluene were mixed in a reacting flask equipped with a Dean-Stark drain device to compose a mixture, heated to 120° C. in a nitrogen atmosphere, and stirred continuously for 22 hours until a yellowish solid was generated. The mixture in the reacting flask was cooled to 80° C. and 200 ml water was added into the mixture. After 10 min stirring, the mixture was filtered to obtain a cake of rough H1 product. The cake was dissolved in 1100 ml of o-xylene solution. The solution was heated to dissolve the cake quickly and then cooled to recrystalize the H1 product. Then, o-xylene solution was filtered to obtain solid H1 product that was further dried at 150° C. Lastly, the solid H1 product was further sublimed to obtain 25 g of H1 yellowish solid in final (yield: 44%).

Physical properties of the compound H1 obtained are: Tg 131.6° C., Tm 329° C.

### Example 2

### Synthesization of H2

First, 10 g of p-phenylenediamine, 112.77 g of 2-iodonaphthalene, 25.94 g of potassium hydroxide, 0.37 g of cupreous chloride, 0.67 g of 1,10-phenanthroline and 185 ml of toluene were mixed in a reacting flask equipped with a Dean-Stark drain device to compose a mixture, heated to 120° C. in a nitrogen atmosphere, and stirred continuously for 22 hours until a yellowish solid was generated. The mixture in the reacting flask was cooled to 80° C. and 200 ml water was added into the mixture. After 10 min stirring, the mixture was filtered to obtain a cake of rough H2 product. The cake was dissolved in 1100 ml of o-xylene solution. The solution was heated to dissolve the cake quickly and then cooled to recrystalize the H2 product. Then, o-xylene solution was filtered to obtain solid H2 product that was further dried at 150° C. Lastly, the solid H2 product was further sublimed to obtain 34 g of H2 yellowish solid in final (yield: 60%).

Physical properties of the compound H2 obtained are: Tg (not detected), Tm 280° C.

### Example 3

### Synthesization of H3

First, 10 g of 1,5-diaminonaphthalene, 77 g of 1-iodonaphthalene, 17.73 g of potassium hydroxide, 0.25 g of cupreous chloride, 0.46 g of 1,10-phenanthroline and 126 ml of toluene were mixed in a reacting flask equipped with a Dean-Stark drain device to compose a mixture, heated to 120° C. in a nitrogen atmosphere, and stirred continuously for 92 hours until a gray-yellow solid was generated. The mixture in the reacting flask was cooled to 80° C. and 150 ml water was added into the mixture. After 10 min stirring, the mixture was filtered to obtain a cake of rough H3 product. The cake was dissolved in 1100 ml of o-xylene solution. The solution was heated to dissolve the cake quickly and then cooled to recrystalize the H3 product. Then, o-xylene solution was filtered to obtain solid H3 product that was further dried at 150° C. Lastly, the solid H3 product was further sublimed to obtain 12.1 g of H3 yellowish solid in final (yield: 29%).

Physical property of the compound H3 obtained is: Tm 373.6° C.

### Example 4

### Synthesization of H4

First, 10 g of p-phenylenediamine, 126 g of 2-iodo-6-methoxyl naphthalene, 25.94 g of potassium hydroxide, 0.37 g of cupreous chloride, 0.67 g of 1,10-phenanthroline and 185 ml of toluene were mixed in a reacting flask equipped with a Dean-Stark drain device to compose a mixture, heated to 120° C. in a nitrogen atmosphere, and stirred continuously for 22 hours until a yellowish solid was generated. The mixture in the reacting flask was cooled to 80° C. and 200 ml water was added into the mixture. After 10 min stirring, the mixture was filtered to obtain a cake of rough H4 product. The cake was dissolved in 1100 ml of o-xylene solution. The solution was heated to dissolve the cake quickly and then cooled to recrystalize the H4 product. Then, o-xylene solution was filtered to obtain solid H4 product that was further dried at 150° C. Lastly, the solid H4 product was further sublimed to obtain 34.5 g of H4 yellowish solid in final (yield: 51%).

### Example 5

### Synthesization of H5

First, 10 g of p-diaminobiphenyl, 66.19 g of 1-iodonaphthalene, 15.23 g of potassium hydroxide, 0.21 g of cupreous chloride, 0.39 g of 1,10-phenanthroline and 135 ml of toluene were mixed in a reacting flask equipped with a Dean-Stark drain device to compose a mixture, heated to 120° C. in a nitrogen atmosphere, and stirred continuously for 30 hours until a yellow solid was generated. The mixture in the reacting flask was cooled to 80° C. and 230 ml water was added into the mixture. After 10 min stirring, the mixture was filtered to obtain a cake of rough H5 product. The cake was dissolved in 1100 ml of o-xylene solution. The solution was heated to dissolve the cake quickly and then cooled to recrystalize the H5 product. Then, o-xylene solution was filtered to obtain solid H5 product that was further dried at 150° C. Lastly, the solid H5 product was further sublimed to obtain 14 g of H5 yellowish solid in final (yield: 37.5%).

Physical properties of the compound H5 obtained are: Tg (not detected), Tm 406° C.

### Example 6

### Synthesization of H17

First, 10 g of 10,10-dibromo-9,9-bianthracene, 12.62 g of N, N-di(2-naphthyl)amine, 4.5 g of sodium tert-butoxide, 50 mg of tris(dibenzylideneacetone)dipalladium (Pd₂(dba)₃), and 33 ml of xylene were mixed in a reacting flask to compose a mixture and heated to 70° C. in a nitrogen atmosphere. Next, 20 mg of tri(tert-butyl)phosphine was added to the mixture. The mixture was gently heated to 140° C., stirred for 3 hours and cooled to 80° C. and then 70 ml of methanol was added into the mixture. After cooling to 25° C., the mixture was filtered to obtain solid H17 product. The solid was dissolved in dimethyl formamide solution and heated for recrystalization. Then, dimethyl formamide solution was filtered to obtain solid H17 product that was further dried at 150° C. Lastly, the solid H17 product was further sublimed to obtain 2 g of H17 yellow solid in final (yield: 12%).

### Example 7

### Synthesization of E1

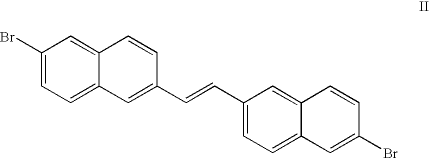

First, 10 g of 11 compound (1,2-bis(6-bromo-naphthalene-2-yl)ethene), 9.3 g of diphenylamine, 5.26 g of sodium tert-butoxide, 60 mg of tris(dibenzylideneacetone)dipalladium (Pd₂(dba)₃) and 38ml of xylene were mixed in a reacting flask to compose a mixture and heated to 70° C. in a nitrogen atmosphere. Next, 30 mg of tri(tert-butyl)phosphine was added into the mixture. The mixture was gently heated to 140° C., stirred for 3 hours and cooled to 80° C. and then 70 ml of methanol was added into the mixture. After cooling to 25° C., the mixture was filtered to obtain solid E1 product. The solid was dissolved in xylene solution and heated for recrystalization. Then, xylene solution was filtered to obtain solid E1 product that was further dried at 160° C. Lastly, the solid E1 product was further sublimed to obtain 5.5 g of E1 solid in final (yield: 39%).

Physical properties of the compound E1 obtained are: Tg 100° C.; Tm 406° C.; and maximum wavelength 451 nm (dissolved in toluene).

### Example 8

### Synthesization of E2

First, 10 g of 11 compound (1,2-bis(6-bromo-naphthalene-2-yl)ethene), 10.8 g of 3,3′-dimethyl-diphenylamine, 5.26 g of sodium tert-butoxide, 60 mg of tris(dibenzylideneacetone)dipalladium (Pd₂(dba)₃) and 38 ml of xylene were mixed in a reacting flask to compose a mixture and heated to 70° C. in a nitrogen atmosphere. Next, 30 mg of tri(tert-butyl)phosphine was added into the mixture. The mixture was gently heated to 140° C., stirred for 3 hours and cooled to 80° C. and then 70 ml of methanol was added into the mixture. After cooling to 25° C., the mixture was filtered to obtain solid E2 product. The solid was dissolved in xylene solution and heated for recrystalization. Then, xylene solution was filtered to obtain solid E2 product that was further dried at 160° C. Lastly, the solid E2 product was further sublimed to obtain 7.8 g of E2 solid in final (yield: 51%).

Physical properties of the compound E2 obtained are: Tg 94° C.; Tm 236° C.; and maximum wavelength 453 nm (dissolved in toluene).

### Examples of Organic Light-Emitting Diodes Containing the Diarylamino Substituted Compounds in the Present Invention

### Example 1

An ITO substrate with a resistivity of 20 Ω/□ was mounted in a vapor-depositing machine. The vapor-depositing machine had a first quartz crucible containing H1, a second quartz crucible containing H5, a third quartz crucible containing 10,10′-di(4-biphenyl)-9,9′-bianthraene, a fourth quartz crucible containing E1, a fifth crucible containing (tris-(8-hydroxyquinolinato)aluminum (Alq₃), a sixth crucible containing aluminum and a seventh crucible containing lithium fluoride.

Pressure in the vapor-depositing machine was reduced to 10⁻⁶ torr. The H1 in the first quartz crucible was heated to a vapor and deposited on the substrate as the hole-injecting layer to a thickness of 55 nm. Then, the H5 in the second quartz crucible was heated to a vapor and deposited on the hole-injecting layer to a thickness of 30 nm to form the hole-transporting layer. Further, a luminescent layer made of 10,10′-di(4-biphenyl)-9,9′-bianthraene was formed on the hole-transporting layer and had a 30 nm thickness containing 3% E1 based on total weight of the luminescent layer. An electron-transporting layer made of Alq3 was formed on the luminescent layer and had a 25 nm thickness. An electron-injecting layer of lithium floride was formed on the electron-transporting layer by vapor deposition and had a 0.7 nm thickness. Lastly, an aluminum cathode membrane with 150 nm thickness was formed on the electron-injecting layer to achieve a first blue-light organic light-emitting diode.

When a direct current of 10 mA/cm² was applied to the first blue light organic light emitting diode, blue light was emitted with a light intensity of 620 cd/m² and a CIE coordinate of (x=0.15, y=0.17). When the light intensity was set at 500 cd/m², the average brightness decreased to half after 1100 hours duration.

### Example 2

A second blue-light organic light-emitting diode has a structure and layer composition the same as the diode in example 1, except 3% weight of E1 was changed to 5% weight of E1 in the luminescent layer (based on the total weight of the luminescent layer). When a 10 mA/cm² direct current was applied to the second organic light-emitting diode, blue light was emitted with a light intensity of 800 cd/m² and a CIE coordinate of (x=0.15, y=0.20).

### Example 3

A third blue-light organic light-emitting diode has a structure and layer composition the same as the diode in example 1, except that the luminescent layer was made of 10,10′-di(4-biphenyl)-9,9′-bianthraene containing 3% weight of E3 (based on the total weight of the luminescent layer). When a 10 mA/cm² direct current was applied to the third organic light-emitting diode, blue light was emitted with a light intensity of 900 cd/M² and a CIE coordinate of (x=0.14, y=0.20). When the light intensity was set at 500 cd/m², the average brightness decreased to half after 1200 hours duration.

### Example 4

A fourth blue-light organic light-emitting diode has a structure and layer composition the same as the diode in example 1, except that the luminescent layer was made of 10,10′-di(4-biphenyl)-9,9′-bianthraene containing 5% weight of E3 (based on the total weight of the luminescent layer). When a 10 mA/cm² direct current was applied to the fourth organic light-emitting diode, blue light was emitted with a light intensity of 1000 cd/m² and a CIE coordinate of (x=0.14, y=0.22).

### Comparison Example 1

A compared blue-light organic light-emitting diode has a structure and layer composition the same as the diode in example 3 of the preferred embodiment in the present invention, except that no hole-injecting layer was formed, the hole-transporting layer was made of NPB (N,N′-Di(naphthalen-1-y)-N,N′-diphenyl-benzidine), and the luminescent layer was made of 10,10′-di(4-biphenyl)-9,9′-bianthraene containing 3% weight of DSA-Ph (based on the total weight of the luminescent layer). When a 10 mA/cm² direct current was applied to the compared organic light-emitting diode, blue light was emitted with a light intensity of 1170 cd/m², and a CIE coordinate of (x=0.15, y=0.26). When the light intensity was set at 500 cd/m², the average brightness decreased to half after 300 hours duration only.

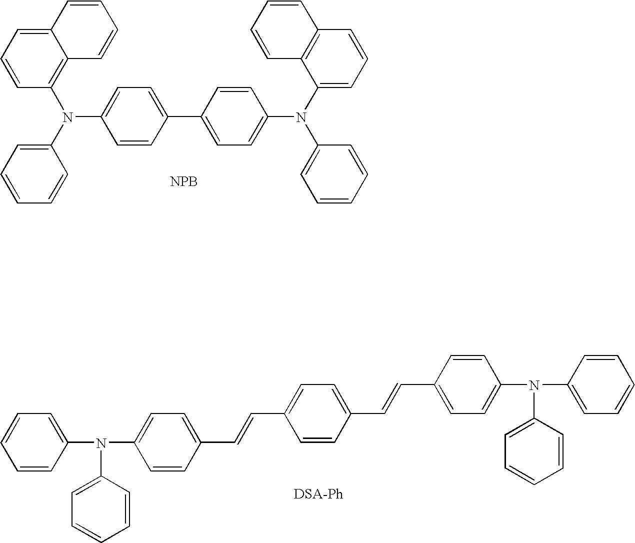

### Example 5

A first green-light organic light-emitting diode has a structure and layer composition the same as the diode in example 1, except that the luminescent layer was made of 10,10′-di(4-biphenyl)-9,9′-bianthraene containing 5% weight of H17 (based on the total weight of the luminescent layer). When a 10 mA/cm² direct current was applied to the first green-light organic light-emitting diode, green light was emitted with a light intensity of 1735 cd/m² and a CIE coordinate of (x=0.25, y=0.61). When the light intensity was set at 500 cd/m², the average brightness decreased to half after 2000 hours duration.

### Comparison Example 2

A compared green-light organic light-emitting diode has a structure and layer composition the same as the diode in example 5 of the preferred embodiment in the present invention, except that the hole-injecting layer was made of TDATA (4,4′,4″-Tris(N-3-methylphenyl-N-phenyl-amino)-triphenylamine), the hole-transporting layer was made of NPB (N,N′-Di(naphthalen-1-yl)-N,N′-diphenyl-benzidine), and the luminescent layer was made of Alq₃containing 1% weight of C-545T (based on the total weight of the luminescent layer). When a 10 mA/cm² direct current was applied to the compared green-light organic light-emitting diode, green light was emitted with a light intensity of 1250 cd/m², and a CIE coordinate of (x=0.31, y=0.62). When the light intensity was set at 500 cd/m², the average brightness decreased to half after 500 hours duration only.

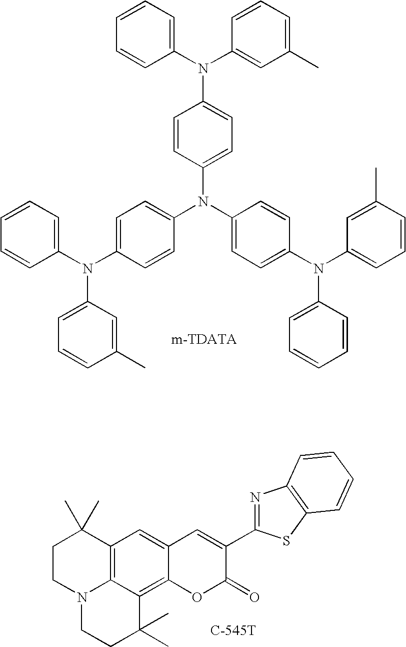

According to the above description, the organic light-emitting diode that contains the diarylamino substituted compounds in the present invention has an excellent luminescent efficiency, high color purity, and most important of all, greatly extended life-time. When the diarylamino substituted compounds are used as dopant within a proper weight proportion range, properties of the organic light-emitting diode are not changed very much so that the diarylamino substituted compounds are suitable for manufacture of large-size substrates in the present OLED panel to reduce defect rate.

The invention has been described in detail with particular reference to certain preferred embodiments. However, variations and modifications can be effected within the spirit and scope of the invention.

Although the invention has been explained in relation to its preferred embodiment, many other possible modifications and variations can be made without departing from the spirit and scope of the invention as hereinafter claimed.

What is claimed is:

1. Diarylamino substituted compounds for electroluminescent devices having the following representative formula (1): 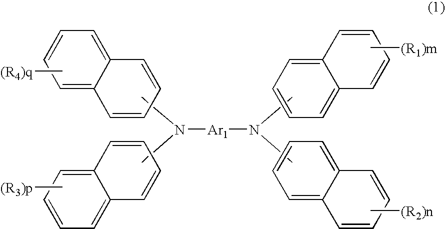
wherein Ar₁ is an aromatic group containing 6-30 carbon atoms; R₁, R₂ R₃ and R₄ are identical or different substituents selected from the group consisting of alkyl containing 1-4 carbon atoms, alkoxyl containing 1-4 carbon atoms, sulfoalkyl containing 1-4 carbon atoms, dialkylamine containing 1-4 carbon atoms and halogen atom; m, n, p and q are integers ranging from 0-2.

2. The diarylamino substituted compounds as claimed in claim 1, wherein Ar₁ is selected from the group consisting of:

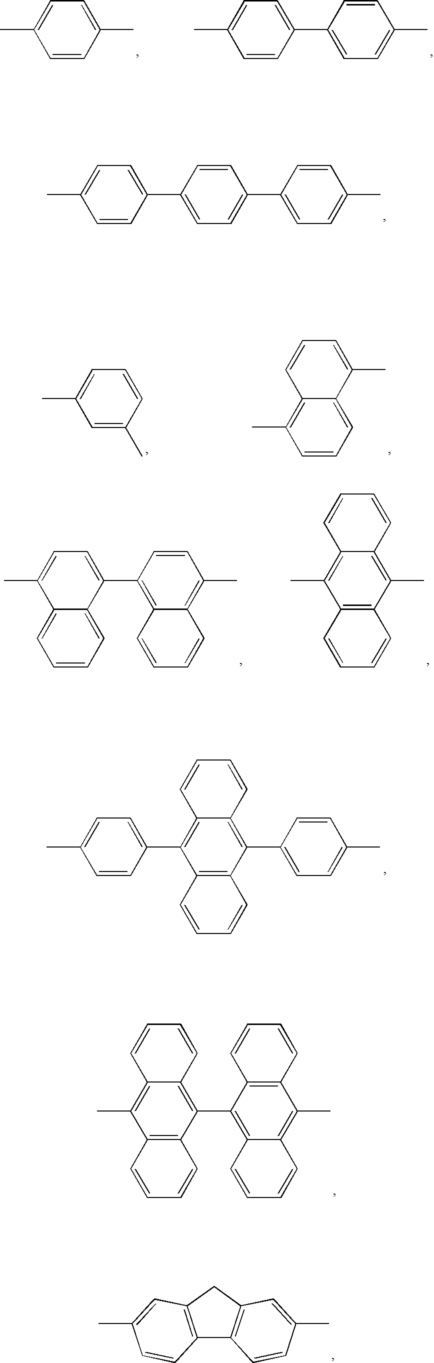

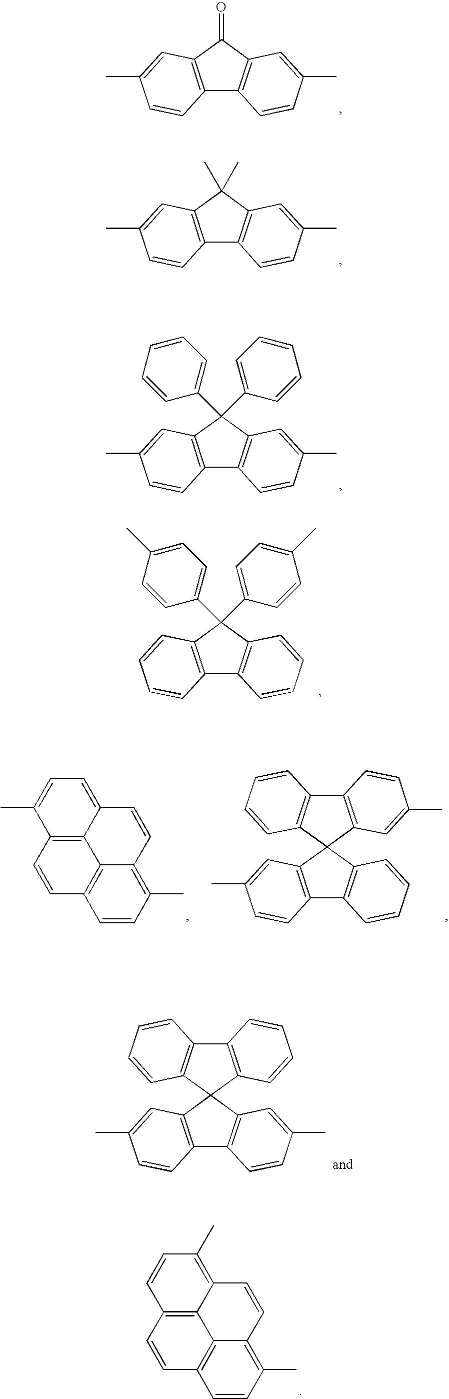

3. The diarylamino substituted compounds as claimed in claim 1, wherein Ar₁ further contains at least one substitute selected from the group consisting of alkyl, alkoxyl, sulfoalkyl, and dialkylamino.

4. The diarylamino substituted compounds as claimed in claim 2, wherein Ar₁ further contains at least one substitute selected from the group consisting of alkyl, alkoxyl, sulfoalkyl, and dialkylamino.

5. The diarylamino substituted compounds as claimed in claim 1, wherein the diarylamino substituted compounds are symmetric in molecular structure.

6. The diarylamino substituted compounds as claimed in claim 2, wherein the diarylamino substituted compounds are symmetric in molecular structure.

7. An organic electroluminescent device using at least one diarylamino substituted compound as claimed in claim 1, wherein the organic electroluminescent device comprises:
an anode;
a hole-transporting layer;
at least one luminescent layer;
an electron-transporting layer; and
a cathode;
wherein, the at least one luminescent layer and the hole-transporting layer comprises at least one of the diarylamino substituted compounds that have the following representative formula (1): 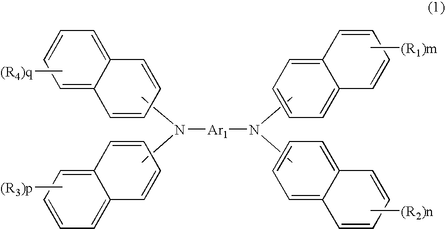
wherein Ar₁ is an aromatic group containing 6-30 carbon atoms; R₁, R₂, R₃ and R₄ are identical or different substituents selected from the group consisting of alkyl containing 1-4 carbon atoms, alkoxyl containing 1-4 carbon atoms, sulfoalkyl containing 1-4 carbon atoms, dialkylamine containing 1-4 carbon atoms and halogen atom; m, n, p and q are integers ranging from 0-2.

8. The organic electroluminescent device as claimed in claim 7, wherein the at least one diarylamino substituted compound is specifically selected from the group consisting of:

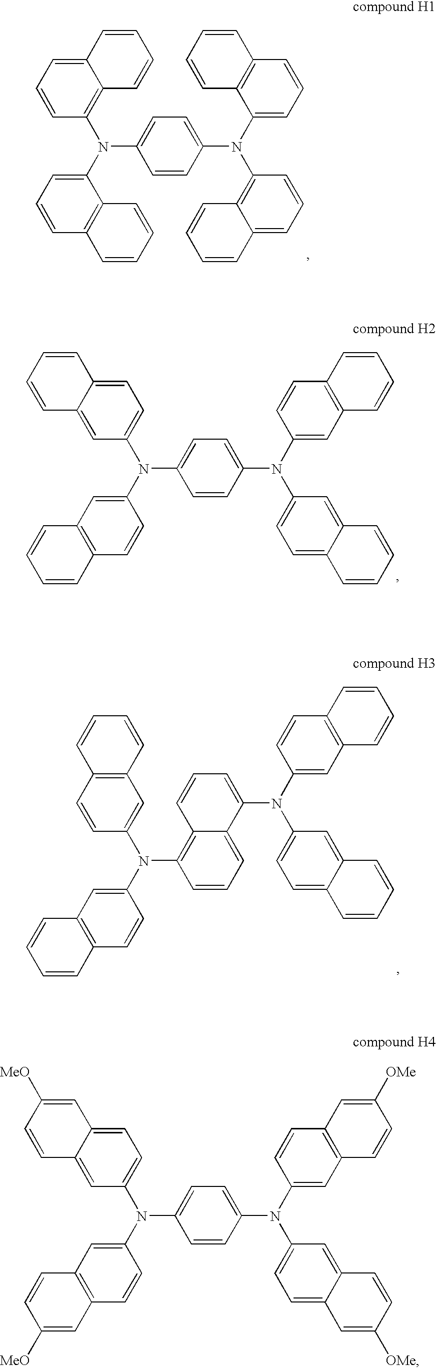

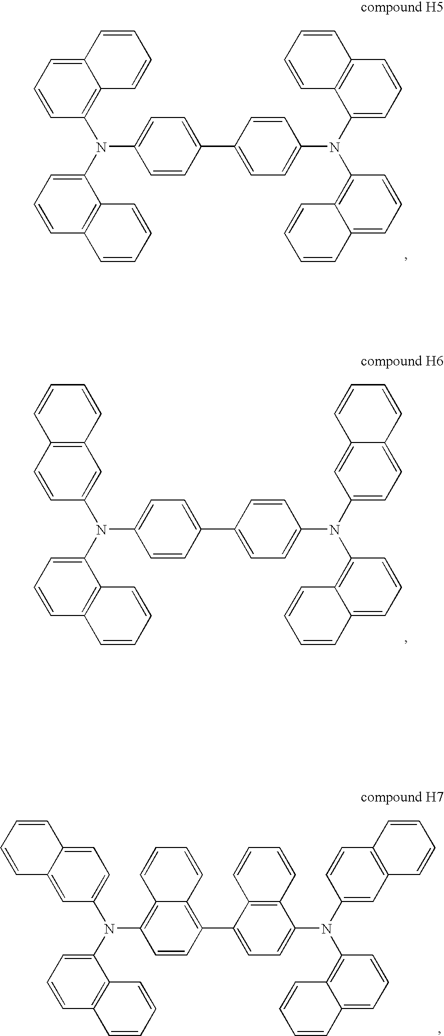

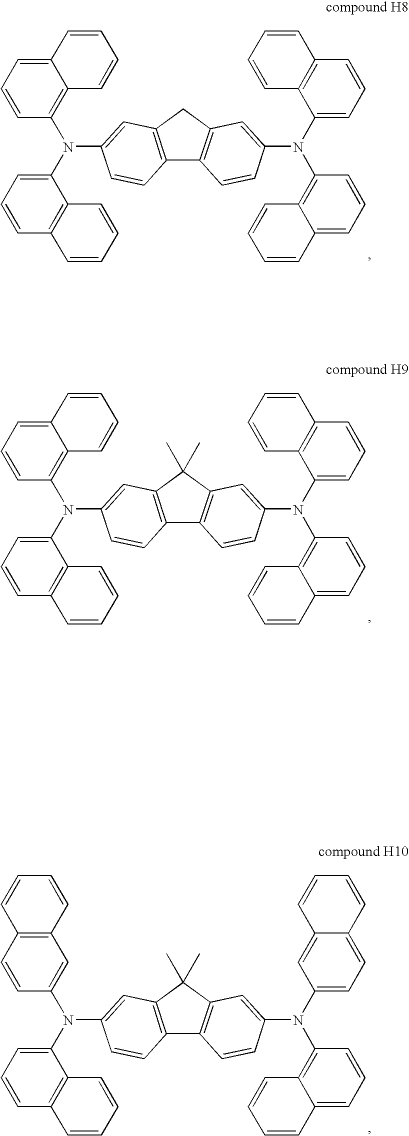

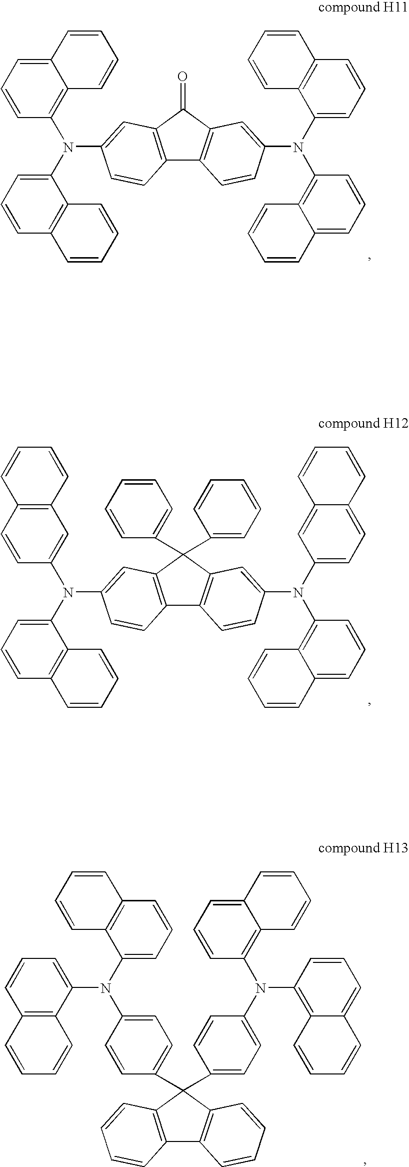

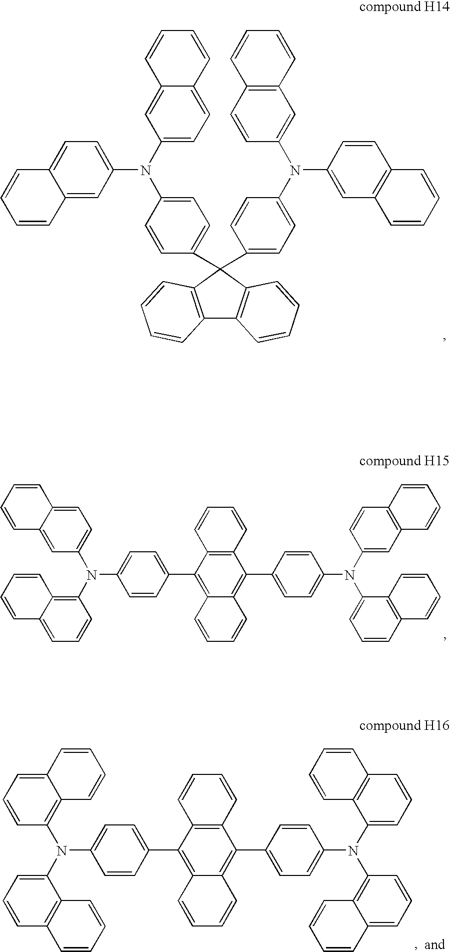

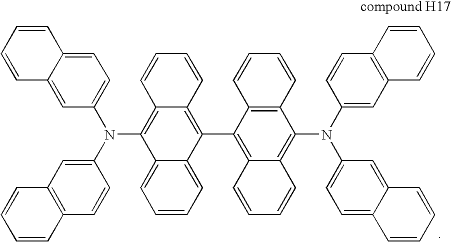

9. The organic electroluminescent device as claimed in claim 7, wherein the luminescent layer comprises at least one compound serving as dopant and having the representative formula (2): 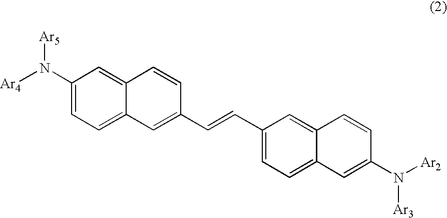
wherein Ar₂, Ar₃, Ar₄, Ar₅ are aromatic groups containing 6-15 carbon atoms.

10. The organic electroluminescent device as claimed in claim 9, wherein the at least one compound of formula (2) is specifically selected from the group consisting of: 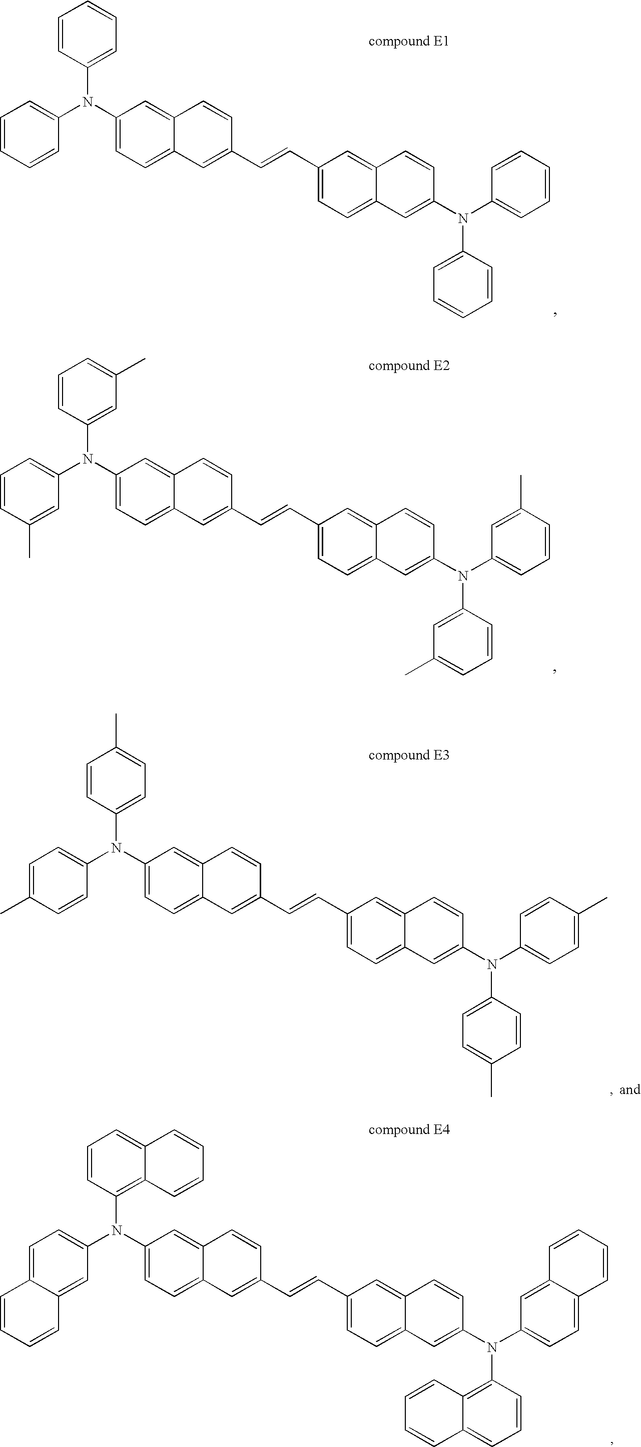
wherein the compound of (2) takes 0.1-50% weight proportion based on a total weight of the luminescent layer.

11. The organic electroluminescent device as claimed in claim 7, wherein the organic electroluminescent device further comprises a hole-injecting layer that contains the at least one diarylamino substituted compound.

12. The organic electroluminescent device as claimed in claim 11, wherein the at least one diarylamino substituted compound is specifically selected from the group consisting of: 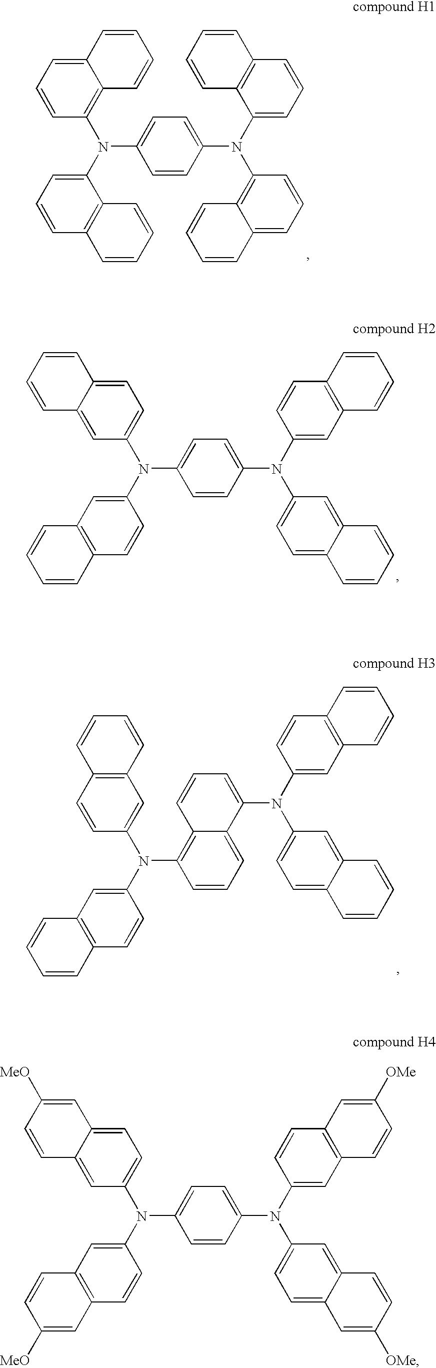 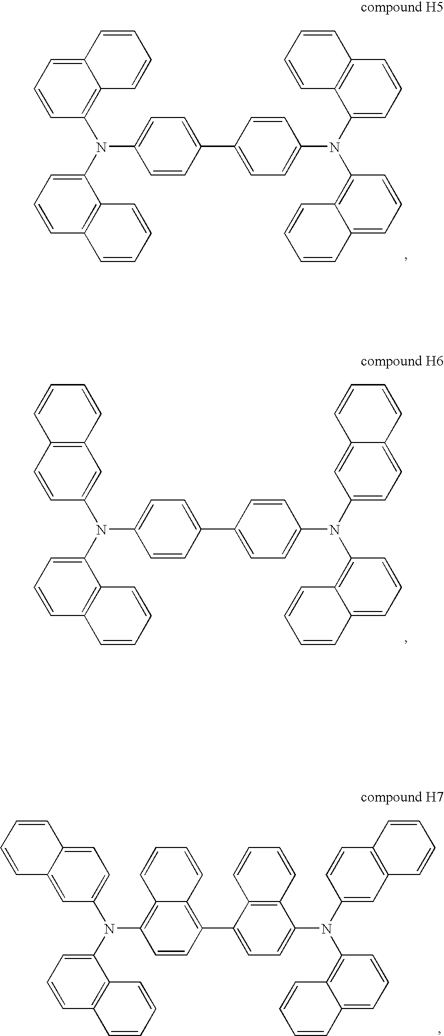 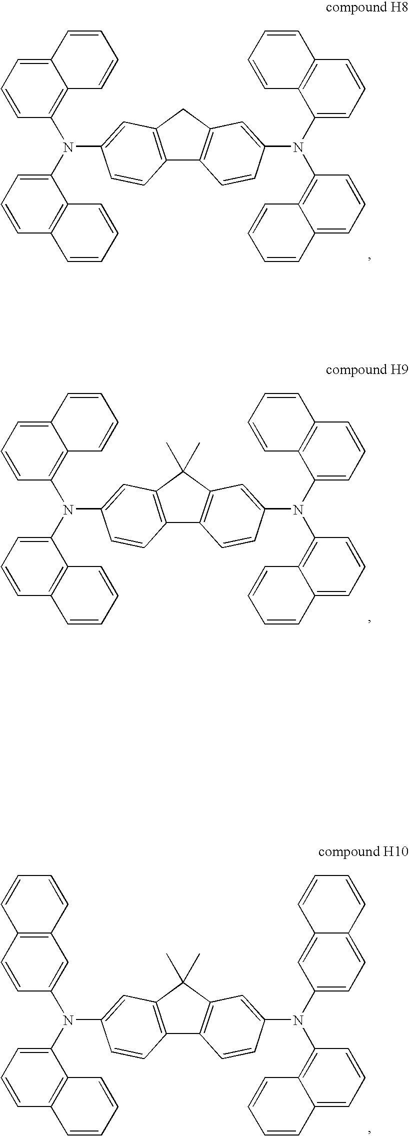 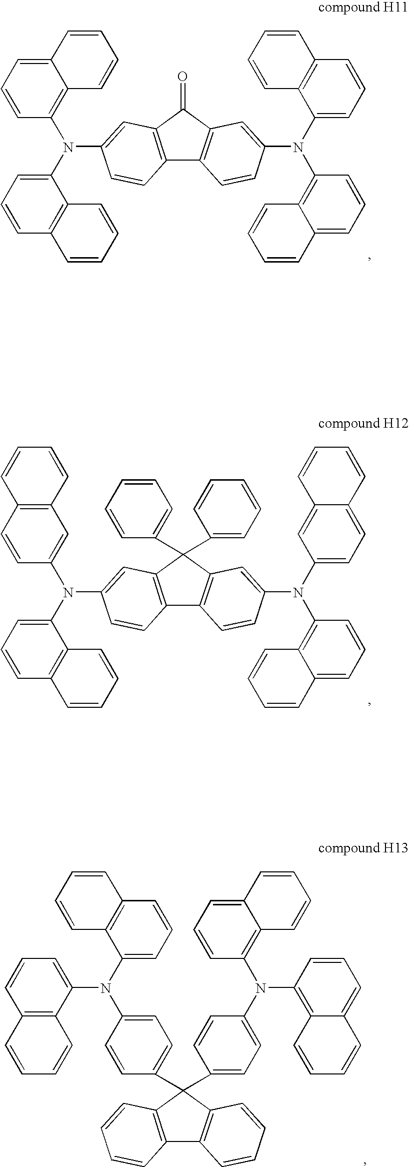 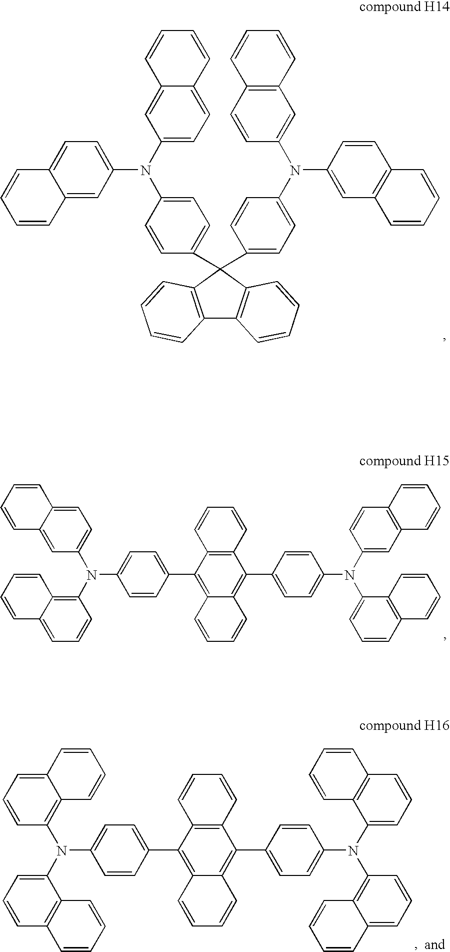 [chemical structure]
